(19) United States
(12) Patent Application Publication
(10) Pub. No.: US 2020/0315832 A1
(43) Pub. Date: Oct. 8, 2020

(54) Removeable Cast Walker With Ankle Lock and Use-Detection and Reporting Means

(71) Applicants: Hadi RAHEMI, Houston, TX (US); Sattar ATASH-BAHAR, Houston, TX (US); Mohammad Sharif SHOURIJEH, Houston, TX (US)

(72) Inventors: Hadi RAHEMI, Houston, TX (US); Sattar ATASH-BAHAR, Houston, TX (US); Mohammad Sharif SHOURIJEH, Houston, TX (US)

(21) Appl. No.: 16/832,621

(22) Filed: Mar. 27, 2020

Related U.S. Application Data
(60) Provisional application No. 62/829,399, filed on Apr. 4, 2019.

(51) Int. Cl. A61F 5/01; G16H 40/67

(52) U.S. Cl. CPC A61F 5/0127; G16H 40/67; A61F 2005/0162; A61F 2005/0153; A61F 2005/0188

(57) ABSTRACT

A range of motion (ROM) removeable cast walker (RCW) has an ankle hinge that may be locked and unlocked by the user. A first sensor is responsive to the locked/unlocked condition of the RCW. A second sensor is responsive to the presence of a limb within the RCW. A third sensor is responsive to weight borne by the RCW. If the ankle hinge is unlocked, the ROM RCW is being worn and weight is being borne, a warning is provided to the patient to remind the patient to lock the ankle hinge to conform to the therapeutic regimen. A radio transmitter may report the state of the ROM RCW to a smart phone and to a remote storage facility for use by a physician.

### CROSS-REFERENCE TO RELATED APPLICATIONS

This application claims priority from U.S. Provisional Application No. 62/829,399, filed on Apr. 4, 2019, which is hereby incorporated by reference.

### BACKGROUND OF THE INVENTION

### 1. Field of the Invention

The present invention generally relates to removeable casts for the foot. More particularly, it relates to “walker boots” having a lockable ankle joint.

### 2. Description of the Related Art

On average, a human leg is lost to diabetes every 30 seconds. The annual cost of diabetic foot ulcers (DFUs) in the United States has been estimated as being at least $15 billion per year. It has been shown that podiatric medical care can reduce the chance of amputation by up to 80%. Offloading the wound—i.e. keeping the patient's weight off the wound—has been shown to be an effective podiatric method in healing DFUs. The gold standard in offloading DFUs is total contact casting (TCC; see FIG. 1) which has the highest and fastest healing rates. However, only 2% of DFUs are treated with TCC due to lack of compliance and discomfort of being in a wound-offloading cast for 1 to 2 months. Removable cast walkers (RCW—FIG. 2) have been shown to be a good alternative for solving the low compliance with TCC. RCW offloading has been shown to produce results comparable to those achieved with TCC when patients use them properly. However, poor adherence to use requirements remains a challenge for RCW treatments.

Both TCC and RCW methods are based on immobilizing the ankle joint, resulting in a decline in peak plantar pressure and therefore offloading the plantar wounds. While ankle locking has been shown to be effective in offloading pressure on the wound, it unfortunately causes the ankle to become stiff (stiffness of connective tissues, e.g. tendons and ligaments) and may result in muscle atrophy. This additional level of stiffness amplifies the higher ankle stiffness already present in patients with diabetic neuropathy (compared to healthy or non-neuropathic diabetic patients). Also, atrophy of the calf muscle when accompanied with sarcopenia in geriatric patients may result in muscle dystrophy.

Increased ankle stiffness after offloading along with lower strength in the calf muscles has been shown to increase plantar pressure and therefore cause more rapid reoccurrence of DFUs. Because of this ankle stiffness, patients may remove an RCW to allow ankle movement. But this can result in the patients forgetting to don the RCW before resuming activities, thus hindering recovery. Range of motion (ROM) RCWs are available to help alleviate the ankle stiffness. In an ROM RCW, the ankle is allowed to pivot to desired limits but then can be locked. While a ROM RCW does allow for improved ankle stiffness, patients forget to lock the hinge when the leave the seated position and begin walking with the hinge unlocked, defeating the purpose of the restraint provided by the RCW.

### BRIEF SUMMARY OF THE INVENTION

A range of motion (ROM) removeable cast walker (RCW) is equipped with an ankle joint that may be locked and unlocked by the user. A first sensor is responsive to the locked/unlocked condition of the ROM RCW. A second sensor is responsive to the presence of a limb within the ROM RCW. A third sensor is responsive to weight borne by the ROM RCW. A radio transmitter may report the output of one or more of the sensors to a remote facility. The output of the sensors may also be recorded in a memory device on the ROM RCW for later downloading.

### BRIEF DESCRIPTION OF THE SEVERAL VIEWS OF THE DRAWINGS

FIG. 1 is an illustration of a total contact cast of the prior art.

FIG. 2 is an illustration of an RCW of the prior art.

FIG. 3 is an illustration of an RCW having range of motion for use according to one embodiment of the invention.

FIG. 4A is an illustration of wear-detection touch sensor locations and weight-bearing pressure sensor location in an embodiment.

FIG. 4B is an illustration of wear-detection capacitive sensor locations in an embodiment.

FIG. 4C is an illustration of stretch sensor locations in an embodiment.

FIG. 5 is an illustration of hinge lock status sensor locations in an embodiment.

FIG. 6 is a representation of capacitive-based hinge lock status sensor operation in an embodiment.

FIG. 7 is a block diagram showing inputs to and outputs from a microcontroller of a ROM RCW according to the invention.

FIG. 8A is an illustration of the rear of a prototype ROM RCW according to the invention showing electronic circuitry, buzzer and battery power pack.

FIG. 8B is an illustration of the side of the prototype ROM RCW shown in FIG. 8A.

FIG. 9 is a flowchart of system operation based on detection units.

### DETAILED DESCRIPTION OF THE INVENTION

Previous efforts to encourage the correct use of RCWs have mainly focused on reminding patients to wear them after they have forgotten to wear them (or elected not to wear them). While these efforts are focused on what happens after the boot has been removed, less attention has been given to the reasons why the boots are removed in the first place. As mentioned before, additional ankle stiffness resulting from ankle locking by TCC and RCW offloading is inevitable. This parameter makes using such remedies uncomfortable and leads to poor adherence, such as patients removing an RCW. ROM RCWs alleviate the ankle stiffness issue but replace it with a basic therapy issue when the patient forgets to lock the ankle when leaving the seated position and beginning walking.

Devices according to the present invention include a ROM RCW with a lockable ankle joint and are able to detect and report whether the boot is being worn, whether the ankle joint is locked or unlocked and the weight-bearing status of the foot. Such devices can create alerting alarms/reminders if the boot is not worn or not worn appropriately (adherence component 1). Allowing the ankle to be relaxed in non-weight bearing conditions may result in more wear time of the boot (adherence component 2) and wearing the ROM RCW may be more pleasant if, in the unlocked condition, the user may control a video game with ankle rotations (adherence component 3).

In a smart ROM RCW according to the present invention, patients may unlock the ankle joint while the joint is not bearing weight—e.g. when the patient is sitting or lying down. This allows the ankle to relax and the soft tissue to be stretched, limiting ankle stiffness. To make wearing the smart ROM RCW even more acceptable (or even exciting) for patients, a gaming feature may be included in the boot, with a sensor in the foot section and mobile app, that may be played by doing ankle plantar flexion and extension. This game-controller feature not only improves adherence to instructions for wearing the smart ROM RCW, but it has two major benefits for wound healing and the remission after healing: A) Ankle exercise increases the rate of circulation and thus provides better delivery of oxygen and nutrients to the ulcer and therefore promotes healing; and, B) Ankle exercise helps to maintain muscle mass and decrease ankle stiffness, thereby promoting faster propulsion and lowering peak plantar pressure after recovery.

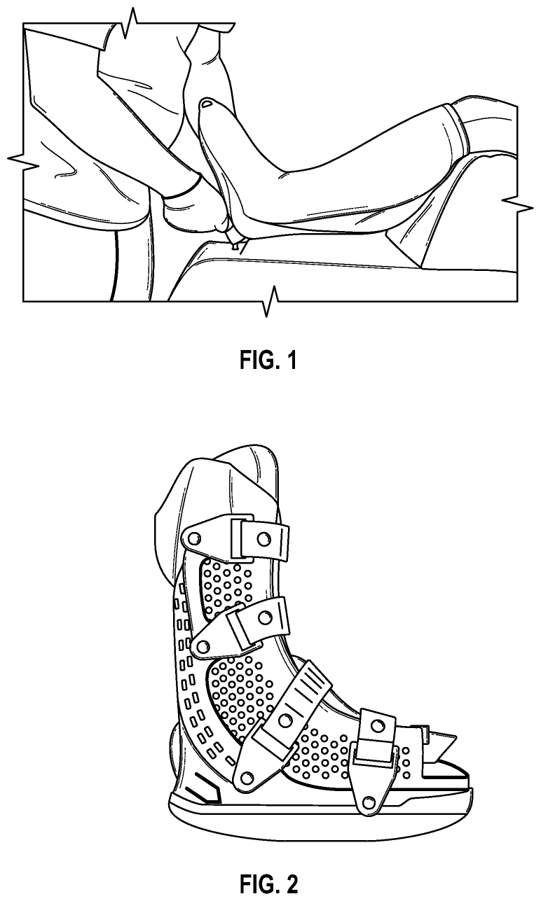

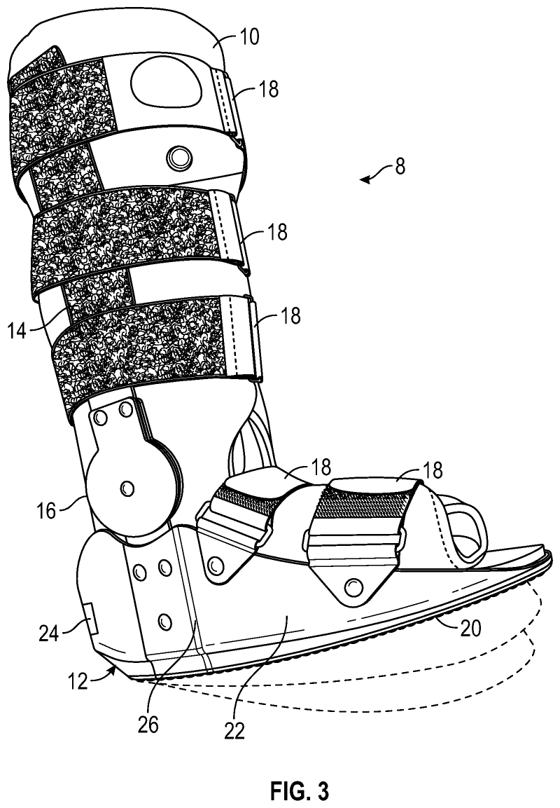

FIG. 3 illustrates a ROM RCW 8 forming the basis of a smart ROM RCW according to the preferred embodiment. The ROM RCW 8 is illustrated as being comprised of a removable preformed lower leg, ankle and foot pad and wrapper 10 adapted to be wrapped about the lower leg, ankle and foot of the patient and to serve as a cushion between the leg, ankle and foot and the orthopedic appliance; a rigid shoe 12 to receive and support the foot of the patient; a pair of lower leg supports or arms 14 on opposite sides of the patient's lower leg (only one of which is visible in FIG. 1); a pair of lockable hinges or hinge assemblies 16 (only one of which is visible) pivotally connecting the lower ends of respective ones of the leg supports 14 to the opposite sides of the shoe at the level of the ankle; and a plurality of adjustable straps 18 for adjustably and releasably securing the walker to the lower leg and foot of the patient.

The pad or wrapper 10 is conventional in the art and is comprised of a polymeric foam cushioning material having a plush or pile exterior surface receptive of the hook members of fabric hook and pile fastening means. The polymeric foam is preferably preformed to conform to and mate with the posterior of the lower leg and the heel and sole of the foot of the patient, and includes opposed flap portions to be wrapped around the anterior surface of the lower leg and the side and upper surfaces of the ankle and the foot. If desired, the inner surface of the exterior or overlying one of the flaps may include hook type fabric fastening means for engagement with the exterior pile surface of the underlying flap to adjustably secure the wrapper about the foot, ankle and lower leg.

The shoe 12 comprises a rigid open top shell, suitably molded from a polymeric resin, having a bottom wall 20, side walls 22 and a heel 24, but no top or upper. Adjacent the heel, the side walls each extend upwardly to adjacent the malleolus of the patient's ankle and each has a semicircular cut-out in the top edge generally conformed to the lower margin of the malleolus. At this point, i.e., from the malleolus down to the sole, each side wall of the shoe is bulged outwardly, as indicated at 26, to form an internal vertical recess for reception of one of the support members or arms of the respective one of the hinges 16. The bottom or sole 20 of the shoe is curved continuously from the heel to the toe to complement the gait of the patient and is covered with a nonslip rubber or composition sole for purposes of safety in walking. The shoe 12 is illustrated in dashed lines in various angles of rotation at the ankle.

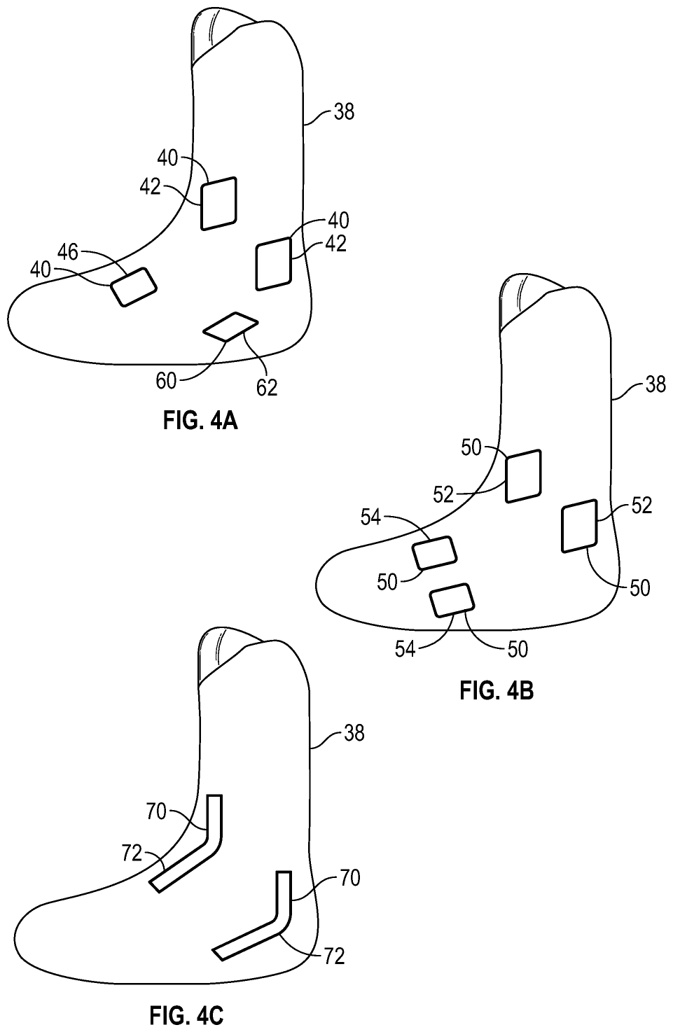

Touch sensors 40 may be provided at locations 42 and 44 as shown in FIG. 4A. Locations 42 are the medial or lateral side of the leg, inside the wrapper 10. Location 46 is in the foot portion of the wrapper 10, either plantar or dorsal. These touch sensors 40 detect the presence of a leg in a smart ROM RCW 38 by use of a proximity sensor which avoids the necessity of direct contact with the skin of the patient, so that the touch sensors 40 are a first type of wear sensor.

Alternatively, the presence of a leg in the smart ROM RCW 38 may be detected using capacitive sensors 50 at locations 52 in both sides of the smart ROM RCW 38 as shown in FIG. 4B or by capacitive sensors 54 at locations 56 on the sole of the smart ROM RCW 38. Locations 52 are both sides of the leg, the medial and lateral sides of the wrapper 10. Locations 56 are in the foot section of the wrapper 10, both plantar and dorsal. Changing the dielectric of this capacitor from air (not worn) to human body (worn) significantly changes the capacitance value which change may be detected, so that the capacitive sensors 50, 54 are a second type of wear sensor.

A pressure sensor 60 at location 62, the proximal side of the foot near the heel as shown or the plantar side of the foot, near the ball, detects if the patient is standing or walking or is seated with no pressure on the foot, so that the pressure sensor 60 is a type of weight-bearing sensor.

In FIG. 4C, stretch sensors 70 are provided at medial and lateral locations 72 inside the wrapper 10, which are from an area below the ankle around to an area level with the ankle. The stretch sensors 70, such as strain gauges, textile-based sensors and polymeric sensors, detect the ankle movement when the hinge is unlocked by the stretching of the wrapper 10, particularly when the patient is walking.

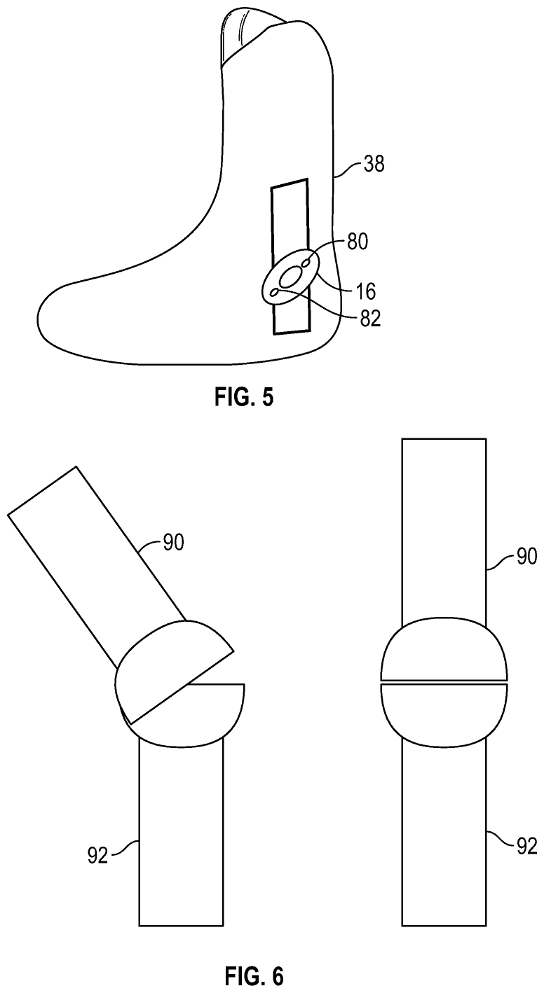

Referring to FIG. 5, pins 80 and 82 lock the hinge 16 in position. In one embodiment, mechanical switches (not shown) are located below or adjacent the pins 80 and 82 to detect if the pins 80 and 82 are installed or removed, thus sensing or allowing a determination if the hinge 16 is locked or unlocked. In another embodiment, a solenoid is used to lock the hinge 16, with the travel of the solenoid indicating the locked state. A mechanical switch can interact with the solenoid for confirmation. In another embodiment, the hinge 16 is locked using a safety key or button, which interacts with a mechanical switch to provide locked state information.

FIG. 6 illustrates an alternative for determining the lock state of the hinge 16. Capacitive sensor portions or elements 90 and 92 are provided on alternate locations of the hinge 16. When the hinge 16 is locked, there is no overlap between the two sensor elements 90 and 92, as shown on the right. The capacitive sensors 90, 92 are located relative to the hinge 16, one capacitive sensor element 90 adjacent a leg support 14 and the other capacitive sensor element 92 adjacent the support of the hinge 16 into the shoe 12. When the hinge 16 is unlocked and the ankle is flexed, the two capacitive sensor elements 90 and 92 have some overlap as shown on the left. Overlapping the two sensor elements 90 and 92 changes the capacitance to allow detection of the unlocked condition.

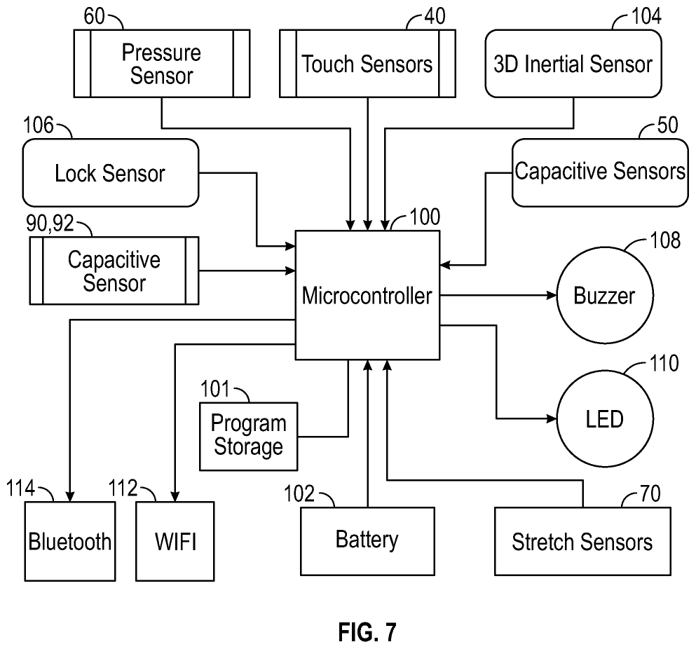

FIG. 7 is a block diagram of a preferred embodiment of the smart ROM RCW. A microcontroller 100, with a connected program storage device 101 containing programs or firmware to control and operate the microcontroller 100. The touch sensors 40, capacitive sensors 50, pressure sensor 60, stretch sensors 70, and capacitive sensors 90, 92 are connected to the microcontroller 100 to provide indications of the wearing state, the lock state and the walking state of the smart ROM RCW. In some embodiments a 3-D inertial sensor 104, such as an accelerometer, gyroscope, and/or magnetic sensor, is connected to the microcontroller 100 to provide alternatives to or enhance the other sensors by detecting movement of the smart ROM RCW or whether the patient is walking, standing or sitting. The 3-D inertial sensor 104 may be placed on the leg and/or foot section of the smart ROM RCW. A buzzer 108 is connected to the microcontroller 100 to provide audio messages, based on the mode of operation or operational status of the boot and particularly including a warning when the user is walking without locking the hinge 16. An LED no connected to the microcontroller 100 is provided for similar visual status messaging, including operational status and warnings, such as low battery condition, communication mode, and the like. A Bluetooth module 114 and a Wi-Fi® module 112 or other radio frequency module are connected to the microcontroller 100 to provide communications capability for the smart ROM RCW to communicate with remote devices, such as smart phones or cloud storage. In one embodiment the Wi-Fi module 112 is used to store and update the state or status of the smart ROM RCW in a cloud-based database for access by both the patient and the physician. In one embodiment the Bluetooth module 114 is used to access the microcontroller 100 for programming, troubleshooting and updating the firmware of the microcontroller 100.

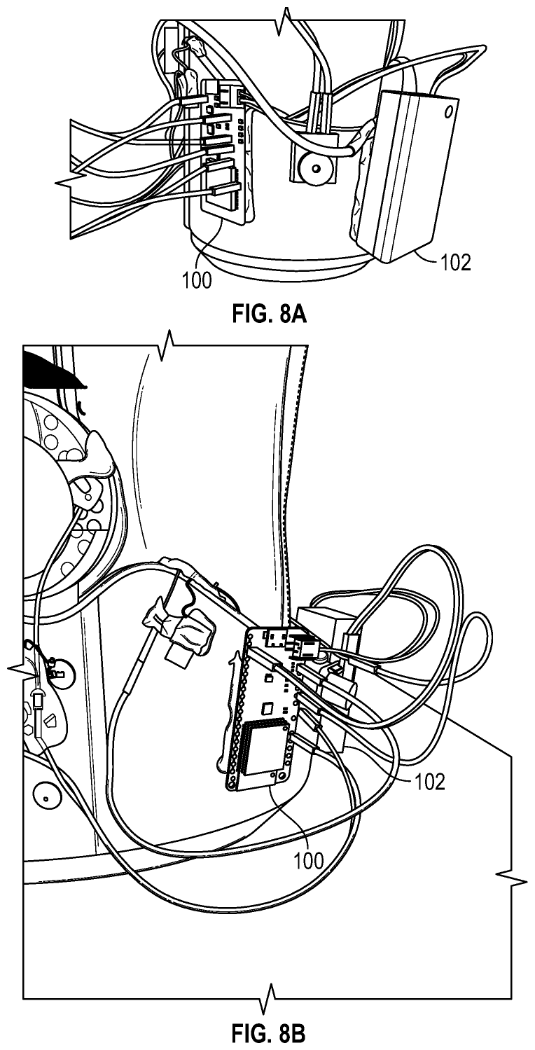

FIGS. 8A and 8B illustrate two views of a prototype embodiment. The microcontroller 100 is a unit such as a Raspberry Pi© or Arduino® while the battery 102 is a larger unit to provide power to the microcontroller 100. The various illustrated wires go to the touch sensors 40, capacitive sensors 50 or 54, pressure sensor 60, stretch sensors 70 and mechanical switch associated with the hinge 16. In a final form, the components are greatly reduced in size and the wiring is fully protected. This allows the components to be mounted to the shoe 12 and the wrapper 10, with a connector to allow the shoe 12 and the wrapper 10 to be disconnected for cleaning and the like. The LED no can be visible from the sides and back of the smart ROM RCW. The buzzer 108 can be mounted to the shoe 12 as well, providing localization of any sounds that are emitted.

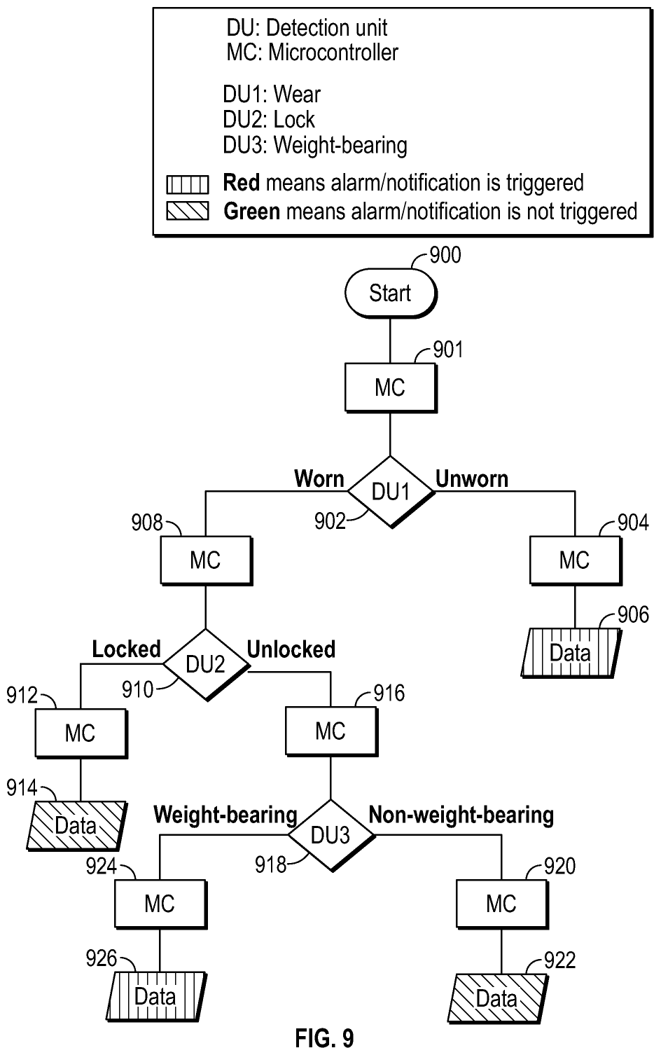

FIG. 9 is a flowchart of operations of a smart ROM RCW according to the present invention, the operations implemented as programs executing on the microcontroller 100. Operation begins at step 900. In step 901, the microcontroller checks the touch sensors 40 or the capacitive sensors 50, depending on the embodiment, to determine if the smart ROM RCW is being worn. In step 902, a determination is made whether the smart ROM RCW is being worn or not worn. If not worn, in step 904 the microcontroller 100 provides a warning 906 that the smart ROM RCW is not worn. This can be a simple red LED no indication or a notification to a smart phone of the patient or caregiver or provided to cloud storage for doctor use. As the smart ROM RCW is not being worn, the states of the other sensors are monitored, so that the sensors are effectively not active.

If the smart ROM RCW is being worn, in step 908 the microcontroller 100 checks the locked state of the smart ROM RCW by checking the lock sensor 106 or the capacitive sensors 90, 92, depending on the embodiment. In step 910, a determination is made if the hinge 16 is locked or unlocked. If locked, in step 912 the microcontroller 100 provides a good indication 914, such as a green LED no indication.

If the hinge 16 is unlocked, in step 916 the microcontroller 100 checks the pressure sensor 60 to determine if the patient is in a weight-bearing state. In step 918, a determination is made whether the patient is in a weight-bearing state. If not, in step 920 the microcontroller 100 provides a good indication 922, such as a green LED no indication. If the patient is in a weight-bearing state, in step 924 the microcontroller 100 provides a warning, such as an audible alarm from the buzzer 108 and a flashing LED no, to alert the patient to the need to lock the hinge 16.

Any change of state, worn or not worn, locked or not locked, weight-bearing or not weight-bearing, can be provided to a patient or caregiver smart phone and to a cloud storage location for use by the doctor.

Table 1 provides the notification state in a table format.

TABLE 1

| Mode of | | | Weight | |
|---|---|---|---|---|
| operation | Lock | Wear | bearing | Notification |
| Mode 0 | True | True | True | No |
| Mode 1 | False | True | True | Yes |
| Mode 2 | True (Not | False | True (Not | Yes |
| | Active) | | Active) | |
| Mode 3 | False (Not | False | True (Not | Yes |
| | Active) | | Active) | |
| Mode 4 | True | True | False | No |
| Mode 5 | False | True | False | No |
| Mode 6 | True (Not | False | False (Not | Yes |
| | Active) | | Active) | |
| Mode 7 | False (Not | False | False (Not | Yes |
| | Active) | | Active) | |

To encourage improved healing, a gaming feature can be incorporated into the smart ROM RCW. Motion sensors, such as inertial sensors, can be implemented in the shoe 12 and active only if the hinge 16 is unlocked and the patient is not bearing weight. Then, with movement of the foot, for example, plantar flexion and extension or rotation at the ankle, signals from the inertial sensor can be used as a game controller for a video gaming system, similar to a keyboard or joystick, to play video games that understand the input mechanism. By controlling a video game by plantarflexion and dorsiflexion of the foot when the ankle joint is unlocked and the boot is not weight-bearing allows for: a) maintaining or lowering the stiffness of the ankle, b) increasing blood flow to the wound for faster healing, and c) decelerating muscle atrophy or dystrophy in older adults that may also be affected by sarcopenia by maintaining muscle volume/quality through exercise.

By determining if the patient is wearing the smart ROM RCW, then determining if the hinge is unlocked and then if the patient is in a weight-bearing state, an alarm or warning is provided to remind the patient to lock the hinge. This allows the use of a smart ROM RCW to alleviate ankle stiffness but at the same time enforce treatment compliance should the patient forget to lock the hinge prior to standing or walking.

The foregoing presents exemplary embodiments of a system embodying the principles of the invention. Those skilled in the art may be able to devise alternatives and variations which, even if not explicitly disclosed herein, embody those principles and are thus within the scope of the invention. Although exemplary embodiments of the present invention have been shown and described, they are not intended to limit what this patent covers. One skilled in the art will understand that various changes and modifications may be made without departing from the scope of the present invention as literally and equivalently covered by the following claims.

What is claimed is:

1. A removable cast walker comprising:
a removable wrapper having portions for positioning around a leg, ankle and foot;
a shoe to receive the foot portion of the wrapper;
a lockable hinge connected to the leg portion of the wrapper and to the shoe, the pivoting point of the hinge being at the ankle joint when worn, the hinge having a rotated position when unlocked;
a wear sensor to provide information on the state of the removable cast walker being worn;
a hinge lock sensor to provide information on the lock state of the lockable hinge; and
a weight-bearing sensor to provide information on the weight bearing state of the removable cast walker.

2. The removeable cast walker of claim 1, further comprising:
a warning indicator; and
a controller coupled to the warning indicator, the wear sensor, the hinge lock sensor and the weight-bearing sensor and configured to activate the warning indicator to provide a warning when the removable cast walker is being worn, the hinge is unlocked and the removable cast walker is bearing weight.

3. The removable cast walker of claim 2, wherein the controller includes a radio frequency module for transmitting removable cast walker state to a remote device.

4. The removable cast walker of claim 3, wherein the remote device is a smart phone.

5. The removable cast walker of claim 4, wherein the smart phone is a warning indicator.

6. The removable cast walker of claim 3, wherein the remote device is cloud storage.

7. The removable cast walker of claim 3, wherein the controller is further configured to act as a gaming controller and provide signals indicative of ankle rotation to a gaming system.

8. The removable cast walker of claim 2, wherein the warning indicator provides an audible warning.

9. The removable cast walker of claim 2, wherein the warning indicator provides a visual warning.

10. The removable cast walker of claim 1, wherein the wear sensor is a capacitive sensor located on the inside of the wrapper and positioned to be activated by either a leg or a foot when the removeable cast walker is worn.

11. The removable cast walker of claim 1, wherein the wear sensor is a touch sensor located on the inside of the wrapper and positioned to be activated by either a leg or a foot when the removeable cast walker is worn.

12. The removable cast walker of claim 1, wherein the hinge lock sensor is a mechanical switch cooperating with a portion of the lockable hinge.

13. The removable cast walker of claim 1, wherein the hinge lock sensor is a capacitive sensor having two portions, the two portions rotating with respect to each other when the hinge is unlocked and rotated and the hinge is locked.

14. The removable cast walker of claim 1, wherein the hinge lock sensor is a stretch sensor that monitors the stretch of the removable wrapper between the locked hinge position and the unlocked and rotated hinge position.

15. The removable cast walker of claim 1, wherein the weight-bearing sensor is a pressure sensor located near the heel or the ball of the foot when the removable cast walker is worn.
